(19) United States
(12) Patent Application Publication
(10) Pub. No.: US 2010/0168147 A1
(43) Pub. Date: Jul. 1, 2010

(54) Medicinal Compositions Comprising Buprenorphine And Naloxone

(75) Inventors: Christopher Bourne Chapleo, Hull (GB); Neil Hyde, Hull (GB)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Reckitt Benckiser Healthcare (UK) Limited, Slough, Berkshire (US)

(21) Appl. No.: 12/529,309

(22) PCT Filed: Feb. 15, 2008
(86) PCT No.: PCT/GB2008/000523

§ 371 Date: Jan. 11, 2010

(30) Foreign Application Priority Data
Mar. 1, 2007 (GB) 0703967.0

(51) Int. Cl. A61K 31/485; A61P 25/04

(52) U.S. Cl. 514/282

(57) ABSTRACT

There is provided a composition for the treatment of pain in human patients wherein said composition comprises buprenorphine to naloxone in a ratio by weight of from 2.1:1 to 8:1, the amount of buprenorphine and naloxone being suitable to provide analgesia, the composition being in a transdermal or transmucosal dosage form. Also provided are an associated method and use.

The present invention relates to medicinal compositions which contain buprenorphine and naloxone and to the use and manufacture of such compositions, as analgesics.

Whilst opioids are particularly effective in the management of moderate to severe pain their use is limited by unpleasant and potentially dangerous adverse effects. Such adverse effects can include sedation, respiratory depression, nausea and gastrointestinal problems. Thus efforts have been made to minimise adverse effects.

There are many opioids and some produce more significant adverse effects than others. Accordingly, careful selection of the opioid employed in an analgesic composition may itself reduce the incidence and severity of adverse effects. One particularly suitable opioid is buprenorphine which has been shown to have both agonist (morphine-like) and antagonist properties without producing significant physical dependence.

Buprenorphine (International Non-proprietary Name for N-cyclopropylmethyl-7[alpha]-[1-(S)-hydroxy-1,2,2-trimethyl-propyl]6,14-endoethano-6,7,8,14-tetrahydronororipavine) is a potent opiate partial agonist analgesic lacking the psychotomimetic effects found with other opiate analgesics. However, buprenorphine suffers from side effects typical of opiate agonists such as nausea and vomiting, constipation and respiratory depression in some patients, although there is a ceiling to its effects on respiratory depression as a direct consequence of its partial agonist properties.

Attempts have also been made to enhance the analgesic effect of opioids while minimising the incidence and severity of adverse effects by combining opioid treatment with other drugs.

One approach is the addition of a non-opioid analgesic to the opioid treatment. The rationale here is that lower levels of opioid should be required to achieve antinociception and thus there should be a reduction of adverse effects.

Another approach is the co-administration of an opioid agonist and low doses of an opioid antagonist.

Given the potent blockade of opioid binding associated with administration of an opioid antagonist it would classically be expected that the use of such an agent would provide no improvement to pain relief and could conceivably increase pain through partial blockade of the agonist it is combined with. It has been found that in some instances antinociception may be potentiated but human studies have generated conflicting findings for the combined use of opioid antagonists and opioid agonists with not all studies being successful.

One such antagonist is naloxone (International Non-proprietary Name for 1-N-allyl-14-hydroxynorhydro-morphinone) which is a narcotic antagonist.

GB2150832 describes analgesic compositions in sublingual or parenteral dosage form comprising an active dose of buprenorphine and an amount of naloxone sufficient to prove aversive, to a narcotic addict by parenteral administration but insufficient to compromise the analgesic action of the buprenorphine. Preferably the parenteral dosage form contains naloxone and buprenorphine within the weight ratio of 1:3 to 1:1 and the sublingual form within the ratio 1:2 to 2:1. The testing in GB-A-2150832 was on rats.

EP 1242087A provides an analgesic composition in parenteral unit dosage form or in a unit dosage form suitable for delivery via the mucosa comprising an amount of buprenorphine which is less than the clinical dose required to achieve pain relief and an amount of naloxone such that the ratio by weight of buprenorphine to naloxone is in the range of from 12.5:1 to 27.5:1, whereby the analgesic action of the buprenorphine is potentiated by the low dose of naloxone. The testing in EP 1242087A was on rats.

Human studies have not been carried out and have generated new findings for the combined use of buprenorphine, as opioid agonist, and naloxone, as opioid antagonist. These new findings extend our understanding of the therapeutic doses which will give effective analgesia in humans.

According to a first aspect of the present invention there is provided a method for the treatment of pain in a human patient, which method comprises transdermal or transmucosal administration to the patient, of buprenorphine and naloxone in the ratio by weight of buprenorphine to naloxone in the range of from 2.1:1 to 8:1.

It is believed that the analgesic action of buprenorphine is potentiated by the achieved naloxone plasma levels, in such modes of administration.

It is to be understood that the terms buprenorphine and naloxone as used herein are intended to cover simple related, pharmaceutically acceptable, compounds such as esters, bases and salts, for example acid addition salts. Particularly preferred salts are the hydrochlorides. However ratios and weights referred to herein refer to buprenorphine and naloxone per se.

Administration may take a few, minutes. Preferably it takes place over a period of at least one minute, preferably at least two minutes, preferably at least three minutes. Preferably it takes place over a period of up to ten minutes, preferably up to seven minutes, preferably up to five minutes.

Suitably, the method comprises transdermal or transmucosal administration to the human patient of buprenorphine and naloxone in the ratio by weight of buprenorphine to naloxone in the range of from 2.2:1 or 2.3:1 or 2.4:1 or 2.5:1 or 3:1 or 3.5:1.

Preferably the method employs transdermal or transmucosal administration to a human patient of buprenorphine and naloxone in a ratio by weight of up to 7.5:1, or 6.8:1, or 6.4:1, or 6:1, or 5.5:1 or 4.5:1. An especially preferred ratio of buprenorphine to naloxone, is 4:1 by weight.

The unit dosage form for transdermal or transmucosal administration may, for example, be a tablet, film, spray, patch, rub-in composition or lozenge. Administration, which will be further described in the second aspect, may comprise the delivery of a medicament comprising buprenorphine and naloxone, preferably in such a form.

Transdermal administration may encompass any mode of administration trough the dermis. Transmucosal administration may encompass any mode of administration trough the mucosa, and sites of administration may include, for example, vaginal and rectal mucosa and, preferably, mucosa of the oral-nasal cavity, for example nasal, throat, buccal and, sublingual sites. Nasal and sublingual administration is especially preferred.

It is preferable to formulate compositions for use in the method in unit dosage forms i.e. physically discrete units containing the appropriate amounts of buprenorphine and naloxone, together with pharmaceutically acceptable diluents and/or carriers; such unit dosage forms being in a form suitable for transdermal or transmucosal administration.

Compositions for use in the method in the form of lozenges and tablets suitably contain soluble excipients selected from materials such as lactose, mannitol, dextrose, sucrose or mixtures thereof. They suitably also contain granulating and disintegrating agents selected from materials such as starch, binding agents such as povidone or hydroxypropyl-methyl cellulose and lubricating agents such as magnesium stearate.

Compositions of the invention may contain a buffer system, for example an organic acid and a salt thereof, such as citric acid and sodium citrate.

The compositions suitable for transdermal or transmucosal administration, as detailed above, may be prepared by manufacturing techniques which are well known to those skilled in the art.

According to a second aspect the present invention provides the use of buprenorphine and naloxone in the manufacture of a medicament for the treatment of pain in a human patient, wherein the medicament is for transdermal or transmucosal administration and the buprenorphine and naloxone are provided in the medicament in a buprenorphine to naloxone ratio by weight of from 2.1:1 to 8:1.

The use of buprenorphine and naloxone in the manufacture of a medicament according to the second aspect may comprise any feature as described in relation to the first aspect.

Thus, preferred ratios of buprenorphine and naloxone in the medicament are preferably as defined above the respect to the first aspect.

In a human being, as stated in EP 1242087B dosages of about 40 μg of buprenorphine per kilogram of body weight are suitably required to obtain satisfactory pain relief in the absence of potentiation. Thus for typical body weights of 50 to 80 kg, the buprenorphine dosage would be from 2 mg to 3.2 mg of buprenorphine per day. This would conveniently be administered as four unit doses.

The amounts of buprenorphine which are required to be effective in the compositions of the invention are less than the amounts which are required to be effective in the absence of the potentiating effects of naloxone.

Importantly when equal doses of buprenorphine with and without the potentiating effect of naloxone are compared, the magnitude and duration of analgesia achieved by the former compositions (i.e. also containing naloxone), are markedly increased. Therefore the same analgesic performance can be achieved with a lower buprenorphine dose when combined with naloxone. It is proposed that an increased analgesic effect can be achieved and/or reduced concentration of buprenorphine can be used, within or across the therapeutic range.

Suitably, unit doses of the compositions of the present invention (containing naloxone) contain buprenorphine in an amount which is below that required to obtain corresponding pain relief in a unit dose of buprenorphine without naloxone.

Suitably, the compositions of the present invention comprise at least 10 μg of buprenorphine per unit dose, preferably at least 15 μg, preferably at least 20 μg, preferably at least 30 μg, and most preferably at least 40 μg. These values reflect the benefit of the invention in achieving analgesia at low dosages.

Suitably, the compositions of the present invention may contain any amount of buprenorphine, up to the upper end of conventional clinical practice. Suitably, they may contain up to up to 32 mg buprenorphine per unit dose, preferably up to 16 mg, preferably up to 8 mg, preferably up to 4 mg, preferably up to 2 mg, preferably up to 1 mg, preferably up to 600 μg, preferably up to 400 μg, preferably up to 200 μg, preferably up to 160 μg, preferably up to 100 μg.

Suitably, in accordance with the present invention, a patient is administered at least 0.25 μg of buprenorphine per kg (of body weight) per 24 hours. Preferably the amount is at least 0.5 μg, preferably at least 1 μg, preferably at least 1.5 μg and most preferably at least 2 μg.

Suitably, in accordance with the present invention, a patient is administered up to 640 μg of buprenorphine per kg per 24 hours. Preferably the amount is up to 320 μg, preferably up to 160 μg, preferably up to 80 μg, preferably up to 40 μg, preferably up to 20 μg, preferably up to 16 μg, and preferably up to 12 μg. Most preferably the amount is not greater than 8 μg.

Suitably by use of compositions of the present invention the amount of buprenorphine administered to a patient for the purpose of achieving relief from pain is at least 40 μg per 24 hours, preferably at least 60 μg, preferably at least 80 μg, preferably at least 120 μg, and most preferably at least 160 μg.

Suitably by use of compositions of the present invention the amount of buprenorphine administered to a patient for the purpose of achieving relief from pain is up to 32 mg, preferably up to 16 mg, preferably up to 8 mg, preferably up to 4 mg, preferably up to 2 mg, preferably up to 1 mg, preferably up to 800 μg, preferably up to 600 μg, preferably up to 400 μg, preferably up to 200 μg, preferably up to 160 μg, preferably up to 100 μg.

Suitably, the composition comprises at least 1 μg of naloxone per unit dose, preferably at least 1.5 μg, preferably at least 2 μg, and most preferably at least 4 μg.

Suitably, the composition comprises up to 4 mg of naloxone per unit dose, preferably up to 2 mg, preferably up to 1 mg, preferably up to 500 μg, preferably up to 300 μg, preferably up to 200 μg, preferably up to 100 μg, preferably up to 80 μg, and most preferably up to 50 μg.

Suitably the amount of naloxone administered is at least 0.025 μg naloxone per kg per 24 hours. Preferably the amount is at least 0.05 μg, preferably at least 0.1 μg, preferably at least 0.15 μg, preferably at least 0.2 μg, and most preferably at least 0.4 μg.

Suitably the amount of naloxone administered is up to 320 μg naloxone per kg of body weight per 24 hours. Preferably the amount is up to 160 μg, preferably up to 80 μg, preferably up to 40 μg, preferably up to 20 μg, preferably up to 10 μg, preferably up to 8 μg, and preferably up to 6 μg. Preferably the amount is not greater than 4 μg per kg of body weight per 24 hours.

Suitably the amount of naloxone administered is at least 5 μg per 24 hours, preferably at least 8 μg, preferably at least 10 μg, preferably at least 15 μg, and most preferably at least 20 μg.

Suitably the amount of naloxone administered is up to 16 mg μg per 24 hours, preferably up to 8 mg, preferably up to 4 mg, preferably up to 2 mg, preferably up to 1 mg, preferably up to 500 μg, preferably up to 400 μg, preferably up to 300 μg, and most preferably up to 200 μg.

References above to the amounts of compounds which may be administered to a patient are with reference to an adult patient.

Whatever the absolute amounts of buprenorphine and naloxone administered, the definition(s) stated herein of the ratio of buprenorphine to naloxone must be satisfied.

According to a third aspect of the present invention there is provided a composition for the treatment of pain in human patients wherein said composition comprises buprenorphine to naloxone in a ratio by weight of from 2.1:1 to 8:1, the amount of buprenorphine and naloxone being suitable to provide analgesia, the composition being in a transdermal or transmucosal dosage form.

Suitably, the composition comprises a medicament as described in the second aspect.

The use of the composition may comprise use in a method according to the first aspect.

The composition according to the third aspect may comprise any feature as described in relation to the first and/or second aspects.

The present invention will now be illustrated by way of example with reference to the following examples.

### Medicament

A sublingual tablet having the following composition:

| | mg/tablet |
|---|---|
| Buprenorphine | 0.08 |
| (as HCl salt) | |
| Naloxone | 0.02 |
| (as HCl salt) | |
| Mannitol | 18.0 |
| Maize starch | 9.0 |
| Povidone | 1.2 |
| Magnesium stearate | 0.45 |
| Lactose | to 60.0 |

was prepared by screening all the materials with the exception of the magnesium stearate through a 750 μm sieve and blending them together. The mixed powders were then subjected to an aqueous granulation procedure and dried at 50° C. The resulting granules were forced through a 750 μm sieve and blended with magnesium stearate (pre-sieved through a 500 μm sieve). The tablet granules were compressed to yield tablets of 5.56 mm diameter and weight 60 mg.

### Nociceptive Testing

The cold pressor (CP) test was used to assess antinociception of buprenorphine and buprenorphine and naloxone combinations administered by retaining the tablet under the tongue so as to dissolve or disperse it (typically after a few minutes) without making efforts to accelerate that process. CP testing was commenced approximately 20 minutes after completion of administration and continued at hourly intervals after that. The compound forms were buprenorphine hydrochloride and naloxone hydrochloride dihydrate. The CP test utilised two plastic cylindrical containers, one of which was filled with warm water and the other with a combination of water and crushed ice to achieve a “slushy” consistency. The subject immersed the non-dominant forearm and hand into the warm water for exactly 2 minutes. At 1 minute 45 seconds, a blood pressure cuff on the immersed arm was inflated to a pressure 20 mmHg below the diastolic blood pressure. The blood pressure cuff minimised the role of blood flow in determining the reaction to cold. At exactly 2 minutes, the forearm was transferred from the warm water to the cold water bath. The subject's eyes were covered for the entire procedure to minimise distraction and cues for time. Upon immersion of the limb in the cold water bath, subjects were asked to indicate when they first experienced pain (pain threshold, CPTHR), then asked to leave their arm submerged until they can no longer tolerate the pain (pain tolerance, CPTOL). Pain threshold and tolerance times were recorded in seconds from immersion in cold. An undisclosed cut-off of 180 seconds was imposed, after which time pain tolerance can no longer be accurately assessed due to numbness. Pain tolerance (CPTOL) is the reported pain response parameter in the current investigations.

For the present tests nociceptive testing was conducted in the same environment, with minimal background noise, audible voices and no clock with audible ticking. Ambient room temperature and lighting was consistent. At no time did the experimenter discuss with the subject his/her performance on the test, or answer any questions related to the average pain tolerance time or any previous results.

The use of these test parameters in a series of double blinded studies allowed the increased magnitude and duration of the analgesia achieved by the combination product compared with that achieved by buprenorphine alone to be demonstrated.

A range of combinations was studied defining the points where the naloxone content was too high and was antagonistic of buprenorphine against analgesia. Additionally the point where the naloxone content was too low and had no synergistic potentiating effect was defined. All naloxone contents between these two points showed beneficial, potentiating effects of naloxone on buprenorphine.

1. A composition for the treatment of pain in human patients wherein said composition comprises buprenorphine to naloxone in a ratio by weight of from 2.1:1 to 8:1, the amount of buprenorphine and naloxone being suitable to provide analgesia, the composition being in a transdermal or transmucosal dosage form.

2. A composition as claimed in claim 1, wherein said ratio is from 2.5:1 to 6:1.

3. A composition as claimed in claim 1, wherein the amount of buprenorphine per dosage unit is from 10 μg to 8 mg.

4. A composition as claimed in claim 1, wherein the composition is adapted for administration to the oral-nasal cavity.

5. A method for the treatment of pain in a human patient, which method comprises transdermal or transmucosal administration to the patient of buprenorphine and naloxone in the ratio by weight of buprenorphine to naloxone in the range of from 2.1:1 to 8:1.

6. A method as claimed in claim 5, comprising sublingual administration.

7. The use of buprenorphine and naloxone in the manufacture of a medicament for the treatment of pain in a human patient, wherein the medicament is for transdermal or transmucosal administration and the buprenorphine and naloxone are provided in the medicament in a buprenorphine to naloxone ratio by weight of from 2.1:1 to 8:1.

8. A method as claimed in claim 5, wherein the administration lasts a period of from 1 minute to 10 minutes.

9. A method as claimed in claim 5, wherein the administration of buprenorphine is in the range 0.25 μg to 640 μg per kg of body weight per 24 hours.

10. (canceled)

11. A composition as claimed in claim 1, wherein said ratio is from 3:1 to 5:1.

12. A composition as claimed in claim 1, wherein said ratio is from 3.5:1 to 4.5:1.

13. A use as claimed in claim 7, wherein the administration lasts a period of from 1 minute to 10 minutes.

14. A use as claimed in claim 7, wherein the administration of buprenorphine is in the range 0.25 μg to 640 μg per kg of body weight per 24 hours.
